(19) United States
(12) Patent Application Publication
(10) Pub. No.: US 2022/0111367 A1
(43) Pub. Date: Apr. 14, 2022

(54) HYDROGENATION CATALYST FOR USE IN HYDROGENATING AMIDE COMPOUND AND METHOD FOR PRODUCING AMINE COMPOUND USING SAME

(71) Applicants: OSAKA UNIVERSITY, Osaka (JP); N.E. CHEMCAT CORPORATION, Tokyo (JP)

(72) Inventors: Kiyotomi KANEDA, Osaka (JP); Takato MITSUDOME, Osaka (JP); Miho KIMURA, Osaka (JP); Yukio TAKAGI, Tokyo (JP); Yoshiyuki WADA, Tokyo (JP)

(73) Assignees: OSAKA UNIVERSITY, Osaka (JP); N.E. CHEMCAT CORPORATION, Tokyo (JP)

(21) Appl. No.: 17/273,118

(22) PCT Filed: Aug. 30, 2019
(86) PCT No.: PCT/JP2019/034075

§ 371 Date: Mar. 3, 2021

(30) Foreign Application Priority Data
Sep. 5, 2018 (JP) 2018-166236

(51) Int. Cl. B01J 27/185; B01J 27/198; C07C 209/26; B01J 37/04

(52) U.S. Cl. CPC B01J 27/1856; B01J 37/04; C07C 209/26; B01J 27/198

(57) ABSTRACT

Provided is a hydrogenation catalyst for an amide compound, containing hydroxyapatite and platinum and vanadium that are fixed on the hydroxyapatite, 15 to 80% of the surface of the platinum being covered with vanadium. The hydrogenation catalyst can promote a reduction reaction in which an amide compound is converted into an amine compound, can be used under mild conditions, and has such durability that the catalyst can be repeatedly used while retaining a high activity.

### TECHNICAL FIELD

The present invention relates to a catalyst for use in a hydrogenation reaction in which an amide compound is converted into an amine compound, the catalyst containing platinum and vanadium that are fixed on hydroxyapatite, and a method for producing an amine compound using the catalyst.

### BACKGROUND ART

A reduction reaction in which an amide compound is converted into an amine compound is one of the most difficult reactions in reduction of carboxylic acid derivatives since amides are resistant to reduction.

In a small scale experiment, like in research, such a reduction reaction in which an amide compound is converted into an amine compound is generally achieved by a method using a strong reductant, such as lithium aluminum hydride (LiAlH₄) or diborane (B₂H₆), on a stoichiometric basis. However, when such a method is used in an industrial scale synthesis, there are problems in that the method causes generation of a large amount of metallic waste and such a reductant, if used in a large amount, is dangerous because of its high reactivity, which leads to generation of hydrogen and the like, and in that a complex post-treatment operation is required.

On the other hand, the reduction reaction from an amide to an amine in which molecular hydrogen is used as a reductant produces only nontoxic water as a byproduct, and thus is an environmentally friendly method for synthesizing an amine. This catalytic hydrogen reduction reaction of amides has been studied for a long period, and has been conducted using cupper-chromium, rhenium, or nickel catalysts. Such a reaction however requires reaction conditions of a high pressure and a high temperature, for example, a hydrogen pressure of 200 atm and a reaction temperature of 200° C. or higher.

In recent years, NPLs 1 and 2 have reported that, when molecular sieve is added to a reaction system, hydrogenation of an amide can be achieved under conditions of a low temperature and a low pressure, for example, 120° C. and 10 atm, or 160° C. and 5 atm. Such a method however has a problem of poor substrate compatibility and producing an alcohol as a byproduct by the C—N cleavage. In addition, such a catalyst cannot be reused.

There are reactions using a homogeneous catalyst as reported in NPL 3, but there is a problem of producing an alcohol as a byproduct by the C—N cleavage. In addition, in a reaction using a homogeneous catalyst, it is difficult to repeatedly use the catalyst which is expensive.

Thus, for industrial use, a catalyst that can be used even under mild conditions and that has such durability that the catalyst can be repeatedly used while retaining a high activity is required.

### CITATION LIST

### Non-Patent Literature

NPT 1: R. Burch, C. Paun, X.-M. Cao, P. Crawford, P. Goodrich, C. Hardacre, P. Hu, L. McLaughlin, J. Sa, J. M. Thompson, Catalytic hydrogenation of tertiary amides at low temperatures and pressures using bimetallic Pt/Re-based catalysts. J. Catal. 283, 89-97 (2011)

NPL 2: M. Stein, B. Breit, Catalytic hydrogenation of amides to amines under mild conditions. Angew. Chem. Int. Ed. 125, 2287-2290 (2013)

NPL 3: E. Balaraman, B. Gnanaprakasam, L. J. W. Shimon, D. Milstein, Direct hydrogenation of amides to alcohols and amines under mild conditions. J. Am. Chem. Soc. 132, 16756-16758 (2010)

### SUMMARY OF INVENTION

### Technical Problem

Accordingly, the present invention has an object to provide a catalyst that can promote a reduction reaction in which an amide compound is converted into an amine compound, that can be used even under mild conditions, and that has such durability that the catalyst can be repeatedly used while retaining a high activity.

### Solution to Problem

As a result of intensive and extensive studies for solving the above problem, the present inventors have found that a catalyst containing platinum and vanadium that are fixed on hydroxyapatite, a certain area of the surface of the platinum being covered with vanadium, has a high hydrogenation activity, selectivity, durability, and reactivity to an amide compound, completing the present invention.

Specifically, the present invention relates to a hydrogenation catalyst for an amide compound, the catalyst containing hydroxyapatite and platinum and vanadium that are fixed on the hydroxyapatite, 15 to 80% of the surface of the platinum being covered with vanadium.

The present invention also relates to a method for producing an amine compound, the method including bringing an amide compound into contact with the hydrogenation catalyst for an amide compound to perform hydrogenation of the amide compound, thus producing an amine compound.

The present invention further relates to a method for producing a hydrogenation catalyst for an amide compound, the method including fixing platinum and vanadium onto hydroxyapatite in a solvent and then drying the resultant.

### Advantageous Effects of Invention

Since the catalyst of the present invention can be used under mild conditions, synthesis of an amine compound from an amide compound can be achieved in a safe and easy manner.

In addition, since the catalyst of the present invention requires no special operation in production, the catalyst can be produced in an inexpensive and safe manner.

Thus, the catalyst of the present invention can be used in an industrial synthesis of an amine compound from an amide compound.

In addition, in the catalyst of the present invention, expensive platinum can be easily collected by filtration after use since the catalyst is fixed on hydroxyapatite, and the collected catalyst can retain the initial activity and selectivity.

Accordingly, the catalyst of the present invention can be readily reused.

### BRIEF DESCRIPTION OF DRAWINGS

FIG. 1 It is a TEM image of Pt—V/HAP which is the catalyst of the present invention.

FIG. 2 It is an ADF-STEM image of Pt—V/HAP obtained in Production Example 1.

FIG. 3 It is a Ca element mapping image of the Pt—V/HAP obtained in Production Example 1.

FIG. 4 It is a V element mapping image of the Pt—V/HAP obtained in Production Example 1.

FIG. 5 It is a Pt element mapping image of the Pt—V/HAP obtained in Production Example 1.

FIG. 6 It is a view of the Ca, V, and Pt element mapping images of the Pt—V/HAP obtained in Production Example 1 which are overlapped.

FIG. 7 It is a graph showing a result of an EDS line analysis of the Pt—V/HAP obtained in Production Example 1.

FIG. 8 It is a graph showing a relationship between the surface coverage and the yield in Example 5.

FIG. 9 It is a graph showing a relationship between the surface coverage and the yield in Example 6.

FIG. 10 It is a graph showing a relationship between the reaction time and the yield in Example 10.

### DESCRIPTION OF EMBODIMENTS

The hydrogenation catalyst for an amide compound of the present invention (hereinafter referred to as “the catalyst of the present invention”) contains hydroxyapatite and platinum and vanadium that are fixed on the hydroxyapatite, 15 to 80% of the surface of the platinum being covered with vanadium. The catalyst of the present invention is herein sometimes denoted as, for example, “X−Y/Z” (X and Y are each a name of metal, such as platinum or vanadium, and Z is hydroxyapatite).

### (Platinum)

Platinum that constitutes the catalyst of the present invention is not particularly limited, but is preferably, for example, platinum particles. As used herein, platinum particles are particles of platinum selected from at least one of metallic platinum or platinum oxide, and are preferably particles of metallic platinum.

Here, the platinum particles are not particularly limited as long as the particles contain platinum, and may contain a small amount of a noble metal, such as ruthenium (Ru), rhodium (Rh), or palladium (Pd). The platinum particles are preferably of metallic platinum. The platinum particles may be primary particles or may be secondary particles. The platinum particles preferably have an average particle size of 1 to 30 nm, and more preferably 1 to 10 nm. Note that the “average particle size” as used herein means an average of diameters obtained by observing any number of particles with an electron microscope.

### (Vanadium)

Vanadium that constitutes the catalyst of the present invention is not particularly limited, but is preferably, for example, vanadium oxide. The vanadium oxide is selected from at least one of vanadate ion (VO₄³⁻, VO₃³⁻), vanadium pentoxide, vanadium (II) oxide, or vanadium (IV) oxide, for example, and is preferably V₂O₅.

### (Molar Ratio of Platinum-Vanadium [Pt—V])

The composition ratio of platinum and vanadium in the catalyst of the present invention in terms of number of moles of platinum (Pt) as metal:number of moles of vanadium (V) as metal (molar ratio [Pt:V]) is 1:0.1 to 10, preferably 1:0.5 to 5, and further preferably 1:0.8 to 1.2.

### (Surface Coverage of Platinum-Vanadium [Pt—V])

In the catalyst of the present invention, 15 to 80%, and preferably 20 to 70% of the surface of the platinum is covered with vanadium. The present inventors have found that the percentage at which the platinum surface is covered with vanadium (referred to as “surface coverage”) has an influence on the hydrogenation activity for an amide. The surface coverage (o) is a numerical value determined by the following (Formula 1). Here, the particle size is an average particle size on the volume basis and can be measured by an EDS line analysis and the like. An optimal surface coverage is about 50%, and it is considered that, in this case, platinum and vanadium are adjacent to each other to provide a competitive catalytic action and each is exposed on the surface to provide a hydrogenation activity for an amide.

$$\begin{matrix}[ Math . 1 ] & \\ Surface coverage ( \% ) = ( 1 - \frac{Particle size of Pt / HAP}{Particle size of Pt - V / HAP} ) \times 100 & ( Formula 1 )\end{matrix}$$

In addition, the surface coverage (%) determined by the above (Formula 1) can be determined by the following (Formula 2) by measuring the adsorption of carbon monoxide (CO) on platinum fixed on hydroxyapatite.

$$\begin{matrix}[ Math . 2 ] & \\ Surface coverage ( \% ) = ( 1 - \frac{CC adsorption on Pt - V / HAP}{CO adsorption on Pt / HAP} ) \times 100 & ( Formula 2 )\end{matrix}$$

The particle size in (Formula 1) is identified by the correlation with the CO adsorption as shown in the following (Formula 3). In (Formula 3), the proportional value is derived from the number of CO molecules that can occupy the specific crystal lattice plane of Pt. In (Formula 1), the proportional value does not appear in the formula due to reduction of the fraction.

$$\begin{matrix}[ Math . 3 ] & \\ Particle size = \frac{Proportional value}{CO adsorption} & ( Formula 3 )\end{matrix}$$

The CO adsorption in the above description can be measured, for example, using a flow-through type chemisorption measurement device. In the flow-through type chemisorption measurement device, CO gas is introduced in a pulse form into a sample cell in which a catalyst is set to measure the adsorption, whereby the active surface area and the particle size of nanoparticles of a noble metal fixed on hydroxyapatite can be measured. CO is selectively adsorbed on the surface of platinum particles, and thus, the area of platinum nanoparticles fixed on hydroxyapatite that are exposed from vanadium can be specified as the CO adsorption. The active surface area of the Pt nanoparticles in Pt—V/HAP is reduced as compared with the active surface area of the Pt nanoparticles in Pt/HAP by the amount corresponding to the Pt nanoparticles that are covered with V. By using the difference, what amount of Pt nanoparticles is covered with V in Pt—V/HAP can be determined. An example of such a flow-through type chemisorption measurement device is BELCAT-A manufactured by BEL JAPAN INC. The conditions for measuring the CO adsorption using this flow-through type chemisorption measurement device are as follows.

### <Measurement Conditions>

The flow rate of carrier gas is always 50 sccm. As a pretreatment, helium gas is allowed to flow at 300° C. for 30 minutes, then the He gas is changed to 02 gas and 02 gas is allowed to flow at 300° C. for 15 minutes, next, He gas is allowed to flow at 400° C. for 45 minutes, and then after cooling to 50° C., He gas is allowed to flow for 10 minutes.

After the pretreatment, the CO adsorption was measured with a composition of the adsorbed gases, CO/He, of 10.05%, at an operation temperature of 50° C., and with an amount of gasper 1 pulse of 100 cm³.

Using this flow-through type chemisorption measurement device under the above conditions, the active surface area and the particle size of Pt nanoparticles in each of Pt/HAP and Pt—V/HAP in which the same amount of Pt is fixed can be measured. The measurement results are assigned in the above (Formula 1) to thereby obtain the surface coverage.

The catalyst of the present invention exhibits excellent performance in a hydrogenation reaction of an amide compound. On the other hand, when the present invention is used in an industrial field, it is sometimes desirable that a more effective use method be selected according to difference in reaction conditions depending on the facilities or the like. For example, when the pressure or temperature is significantly low in the reaction, a lower surface coverage of the catalyst may be desirable. In a catalyst with a lower surface coverage, the area of platinum exposed on the surface of the catalyst particles is larger to increase the active points in the catalyst surface. Such a catalyst having many active points is expected to rapidly promote hydrogenation of an amide compound even under adverse conditions in terms of promotion of the reaction. When the catalyst of the present invention is used in such a reaction under significantly mild conditions, the surface coverage may be preferably 15 to 40%, and more preferably 15 to 30%.

In contrast, in the case of a higher pressure or temperature in the reaction, if platinum which forms active points is largely exposed on the catalyst surface, a reaction that proceeds with platinum alone, such as hydrogenation of carbon-carbon double bond, is preferentially promoted, which possibly leads to a decrease in the yield of hydrogenation of an amide compound which is the target of the present invention. In this case, for the purpose of suppressing the reaction by platinum alone, it is sometimes desirable to use a catalyst in which the surface coverage is set to a higher value. Thus, when the catalyst of the present invention is used in a reaction under such conditions that generally promote a catalytic reaction, the surface coverage may preferably be 50 to 80%, and may more preferably be 60 to 80%.

### (Hydroxyapatite)

The hydroxyapatite (base material) of the catalyst of the present invention is not particularly limited, and, for example, the so-called BET value (specific surface area value) can be used as an index of the adsorption ability. The BET value may be 0.1 to 300 m²/g, and the average particle size may be 0.02 to 100 μm. In the present invention, the adsorption ability of the hydroxyapatite is preferably 0.5 to 180 m²/g.

In addition, the form of the hydroxyapatite is not particularly limited, and examples of the form include a powder form, a spherical particle form, an amorphous granular form, a cylindrical pellet form, an extruded shape, and a ring shape.

The hydroxyapatite is not particularly limited, and includes, not only calcium hydroxyphosphate having a general stoichiometric composition, Ca₁₀(PO₄)₆(OH)₂, but also a calcium hydroxyphosphate compound having a composition similar to the above composition, tricalcium phosphate, and the like.

In the catalyst of the present invention, the aspect of platinum and vanadium fixed on the hydroxyapatite is not particularly limited, and various aspects can be employed according to the form of the hydroxyapatite. The position of fixing does not have to be simply controlled, and may be on the inside of pores or a layer or may be only on the surface. However, it is preferred that platinum having a small particle size be fixed in a dispersed manner and vanadium be present in the vicinity of the platinum or on the platinum. Note that the amount of platinum and vanadium oxide fixed on the hydroxyapatite in the catalyst of the present invention is not particularly limited, but, for example, the amount of platinum as metal is preferably 0.1 to 10% by weight.

Since the catalyst of the present invention uses such a hydroxyapatite as described above, separation is easily achieved after the catalyst is used in a reaction, and the catalyst is obviously advantageous in terms of reuse of the catalyst.

(Component that can be Added to the Catalyst)

The catalyst of the present invention may be any catalyst in which platinum and vanadium are fixed on hydroxyapatite. A transition metal, an alkali metal, an alkaline earth metal, or the like may be incorporated as a component of the catalyst or a component of the hydroxyapatite according to an ordinary method to the extent that the effect is not impaired.

### (Method for Producing the Catalyst of the Present Invention)

The catalyst of the present invention can be basically produced by fixing platinum and vanadium on hydroxyapatite in a solvent, and then drying the resultant (hereinafter referred to as “the inventive method”). In the catalyst of the present invention, the percentage at which the platinum surface is covered with vanadium can be adjusted by adjusting the amounts and the ratio of amounts of a platinum compound and a vanadium compound contained in a solvent mixture liquid in a production method as described later.

### (Amounts of Platinum Compound and Vanadium Compound Used)

The amounts of the platinum compound and the vanadium compound used (amounts of the compounds charged) in preparation of the catalyst of the present invention are not particularly limited, but regarding the platinum compound and the vanadium compound, the ratio [number of moles of vanadium as metal/number of moles of platinum as metal] is preferably 0.14 to 2.4, and more preferably 0.3 to 2.

### (Amount of Platinum Compound and Vanadium Compound Fixed)

When the amount of platinum and vanadium fixed in the catalyst of the present invention per unit weight of the hydroxyapatite as a carrier is too small, the dispersibility of platinum and vanadium is too high and it may be difficult to cover the platinum surface at an appropriate coverage. In the present invention, the amount of the compounds fixed in terms of the [(number of moles of vanadium as metal+number of moles of platinum as metal)/weight of hydroxyapatite] is preferably 0.4 to 1.4 mmol/g, and more preferably 0.5 to 1.2 mmol/g.

In the inventive method, a specific method for fixing platinum and vanadium on hydroxyapatite in a solvent is not particularly limited, and examples thereof include a method of mixing hydroxyapatite and a solvent mixture liquid containing a platinum compound and a vanadium compound to fix platinum and vanadium on the hydroxyapatite in a solvent, and a method of sequentially mixing hydroxyapatite, a solvent liquid containing a platinum compound, and a solvent liquid containing a vanadium compound in any order to fix platinum and vanadium on the hydroxyapatite in a solvent.

The platinum compound used in the inventive method is not particularly limited, but is preferably a compound that forms platinum particles on hydroxyapatite upon drying. Examples of the platinum compound include a platinum complex salt, such as platinum acetylacetonate (Pt(acac)₂), tetraammineplatinum(II) acetate, dinitroammine platinum(II), hexaammine platinum(IV) carbonate, or bis(dibenzalacetone)platinum (0), and a salt, such as platinum chloride, platinum nitrate, or potassium tetrachloroplatinate, with Pt(acac)₂ being particularly preferred.

The vanadium compound used in the inventive method is not particularly limited, but is preferably a compound that produces a vanadium oxide on hydroxyapatite upon drying. Examples of the vanadium compound include a vanadium complex salt, such as vanadyl acetylacetonate (VO(acac)₂) or tetramethylammonium bis(tartrato)bis[oxovanadate(IV)], and a salt, such as ammonium vanadate(V) or vanadium naphthenate, with VO(acac)₂ being particularly preferred.

The solvent mixture liquid containing a platinum compound and a vanadium compound used in the inventive method is a liquid in which the platinum compound and the vanadium compound are suspended in a solvent. The molar ratio of the platinum compound and the vanadium compound in the solvent mixture liquid is 1:0.1 to 10, preferably 1:0.5 to 5, and further preferably 1:1. Examples of the solvent include water and an organic solvent, such as an alcohol or acetone. Water, which is superior in the cost and safety, is preferred. Such a solvent may be used alone or two or more of such solvents may be used in combination. The temperature of the solvent is not particularly limited, but, for example, the temperature is 0 to 100° C., and preferably 10 to 50° C.

The solvent mixture liquid prepared as described above may then be mixed with hydroxyapatite. The method for mixing the solvent mixture liquid and hydroxyapatite is not particularly limited as long as such amounts that all the components are sufficiently dispersed are used. Hydroxyapatite in an amount of 0.1 to 100 g, and preferably 1 to 10 g relative to 0.1 mmol of platinum as metal is used with stirring. Stirring is continued after mixing for 0.5 to 12 hours, preferably for 1 to 6 hours.

A solvent liquid containing a platinum compound and a solvent liquid containing a vanadium compound used in the inventive method are a liquid in which the platinum compound and the vanadium compound are each suspended in a solvent. The contents of the respective compounds in the solvent liquids may be made so that the contents thereof obtained after mixing the solvent liquids are the same as the contents thereof in the above solvent mixture liquid containing the platinum compound and the vanadium compound. In addition, the solvent used therein and the temperature of the solvent may be the same as in the above solvent mixture liquid.

Using the solvent liquid containing a platinum compound and the solvent liquid containing a vanadium compound which are prepared as described above, next, hydroxyapatite, the solvent liquid containing a platinum compound, and the solvent liquid containing a vanadium compound may be mixed in any order. An order in which the hydroxyapatite and the solvent liquid containing a platinum compound are mixed first and then the mixture is mixed with the solvent liquid containing a vanadium compound is advantageous in that a transition metal tends to be fixed on a platinum compound. An order in which a platinum compound is mixed later is advantageous in that the loss of the expensive platinum may be reduced. The method for mixing the solvent liquid and hydroxyapatite may be the same as in the case of using the solvent mixture liquid.

After the solvent mixture liquid and the hydroxyapatite are mixed or both the solvent liquids and hydroxyapatite are mixed to fix platinum and vanadium on hydroxyapatite in a solvent as described above, the resultant may be dried. Before drying, a pretreatment including washing, filtration, concentration, and the like is preferably performed to remove the solvent. The condition of drying is not particularly limited, but, for example, drying is performed at 80 to 200° C. for 1 to 56 hours. After drying, for example, the catalyst is preferably subjected to calcination using a muffle furnace or the like at 250 to 700° C. for 1 to 12 hours or other treatments, and the resultant may further be subjected to pulverization or the like.

Examples of the platinum compound in the case where water is used as a solvent in the inventive method include a platinum salt, such as a hexachloroplatinic(IV) acid (H₂PtCl₆) or a tetrachloroplatinic(II) acid salt (K₂PtCl₄, etc.). Among them, potassium tetrachloroplatinate(II) (K₂PtCl₄) is preferred. Examples of the vanadium compound include a vanadium salt, such as vanadium chloride (VCl₃), and a vanadic acid salt, such as sodium metavanadate (NaVO₃), sodium orthovanadate(V) (Na₃VO₄), potassium metavanadate (KVO₃), or ammonium metavanadate (NH₄VO₃). Among them, vanadium chloride is preferred.

In addition, when water is used as a solvent in the inventive method, if the compound is difficult to dissolve in the solvent, a pH adjuster, a binder, or the like may be used, ultrasonic waves may be applied, or the temperature may be adjusted, to the extent that the catalytic performance is not impaired. Examples of the pH adjuster include sodium hydroxide, sodium carbonate, sodium hydrogen carbonate, potassium carbonate, potassium hydrogen carbonate, ammonia, acetic acid, citric acid, carbonic acid, and lactic acid. Examples of the binder include an organic compound, such as polyethylene glycol or polyvinyl alcohol, and an inorganic compound, such as silica.

In the catalyst of the present invention, platinum and vanadium (hereinafter simply referred to as “platinum, etc.”) may be uniformly fixed in hydroxyapatite particles or may be biasedly fixed on the surface side of hydroxyapatite. Regarding the position of fixing platinum, etc., it is desirable that the component be biasedly fixed on the surface side of hydroxyapatite, particularly when expensive components, such as platinum, etc., are to be effectively used. Biasedly fixing on the hydroxyapatite surface increases the chance of a reaction substrate to come in contact with platinum, etc., whereby an increase in the catalytic activity is expected.

The method for biasedly fixing platinum, etc. on the hydroxyapatite surface is not particularly limited, and can be appropriately selected from known techniques according to the catalyst material used. Specific examples thereof include a technique in which the pH of the solvent mixture liquid containing a platinum compound and a vanadium compound or the pH's of the solvent liquid containing a platinum compound and the solvent liquid containing a vanadium compound is (are) adjusted, a method in which, for insolubilizing (precipitating) platinum, etc. on hydroxyapatite, a treatment with an aqueous solution used for insolubilization, such as an aqueous alkali solution, is performed to immobilize the platinum, etc. before or after mixing the solvent mixture liquid or the solvent liquids with hydroxyapatite, a technique in which the temperature and the settling time are controlled after mixing the solvent mixture liquid or the solvent liquids with the hydroxyapatite to perform aging, and a technique in which a calcination step is further added after producing the catalyst of the present invention. Note that in the above techniques, washing, drying, and the like may be appropriately performed.

In the technique of adjusting the pH(s) of the solvent mixture liquid or the solvent liquids, the pH adjuster as described above can be used. The pH(s) of the solvent mixture liquid or the solvent liquids may be adjusted using the pH adjuster so that fixing on hydroxyapatite is easily achieved. The pH may be made acidic, alkaline, or neutral.

In the technique in which a treatment with an aqueous solution used for insolubilization, such as an aqueous alkali solution, is performed before or after mixing the solvent mixture liquid or the solvent liquids with hydroxyapatite, an aqueous alkali solution in which an alkaline compound is dissolved in water or the like is used. Examples of the alkaline compound include a hydroxide of an alkali metal or an alkaline earth metal, a bicarbonate of an alkali metal or an alkaline earth metal, a carbonate of an alkali metal or an alkaline earth metal, a silicate of an alkali metal or an alkaline earth metal, and ammonia. In addition, the pH in this case is not particularly limited, but is 7 to 14, and preferably 8 to 13.

Regarding the amount of the aqueous alkali solution used in the treatment for insolubilization, since the purpose is to immobilize a platinum compound and a vanadium compound, an aqueous alkali solution having a concentration adjusted so that the amount of alkali is slightly excess, for example, 1.05 to 1.2 times, relative to the substance to be reduced is preferably used.

In the technique of performing aging, the temperature and the settling time after mixing the solvent mixture liquid or the solvent liquids with the hydroxyapatite may be appropriately set and are not particularly limited, but, for example, ageing may be performed at 10 to 100° C. for 1 to 72 hours, and preferably at 30 to 70° C. for 2 to 24 hours.

In a technique in which a calcination step is further added after producing the catalyst of the present invention, the produced catalyst of the present invention may be calcined while applying a heat reduction treatment in a gas atmosphere containing hydrogen. Such calcination is also referred to as gas phase reduction or hydrogen reduction. In the case of gas phase reduction, there is no solvent that mediates the reduction and the component to be reduced is difficult to move to make it difficult for particles of the platinum, etc. to aggregate, thus making it possible to fix the platinum, etc. in the form of small particles.

When the calcination step is performed, the platinum, etc. is sometimes oxidized through calcination. In this case, a reduction treatment is preferably applied. In the reduction treatment, gas phase reduction and liquid phase reduction can be employed. In the gas phase reduction, a reductive gas is supplied to the catalyst heated to 100 to 500° C. to apply a reduction treatment. As the reductive gas, in addition to hydrogen as described above, carbon monoxide or a low molecular weight hydrocarbon may be used. As the low molecular weight hydrocarbon, methane, ethane, propane, butane, ethylene, and the like can be used. In the case of gas phase reduction, the composition of the gas phase used may be constituted only of a reductive component, or a composition in which a reductive component is mixed with a gas that is inactive in reduction, such as nitrogen, may be used.

In the liquid phase reduction, a reductive liquid and the catalyst are mixed and heated to 80 to 150° C. to reduce an oxidized catalyst component. The reductive component used is not particularly limited, and may be appropriately selected depending on the reduction conditions. Examples thereof include formic acid, sodium formate, and hydrazine.

The thus obtained catalyst of the present invention contains hydroxyapatite, and platinum and vanadium fixed thereto, 15 to 80% of the surface of platinum being covered with vanadium.

Note that the production of the catalyst of the present invention may be confirmed, for example, in a manner that fixing of platinum and vanadium on hydroxyapatite is confirmed using a transmission electron microscope (TEM), a field emission-scanning electron microscope (FE-SEM), energy dispersive x-ray spectroscopy (EDX), or the like. Furthermore, the surface coverage may be determined in the method as described above to check whether the value falls in the above range.

### (Hydrogenation of Amide Compound)

The catalyst of the present invention is for a hydrogenation reaction of an amide compound. Thus, when the catalyst of the present invention is brought into contact with an amide compound, the amide compound can be hydrogenated (reduced) to produce an amine compound.

The amide compound is not particularly limited as long as it is a compound having an amide bond, but, for example, a secondary or higher amide compound, an amide compound having an aromatic substituent, an amide compound in which two substituents not including the carbonyl bonded to the N atom in a lactam or tertiary amide are bonded to each other to form a cyclic structure, or the like is preferred, and a secondary or higher amide compound or an amide compound having an aromatic substituent is more preferred. Note that the amide compounds in the present invention include imide compounds.

The method for bring the catalyst of the present invention into contact with an amide compound to hydrogenate the amide compound is not particularly limited and may be appropriately selected. Specifically, in a pressure resistant vessel, such as an autoclave, the catalyst of the present invention and an amide compound are brought into contact with hydrogen gas in a liquid phase to thereby cause hydrogenation of the amide compound. In addition, in the hydrogenation, molecular sieve or the like may be placed in the vessel for removing water to promote the reaction. Furthermore, the catalyst of the present invention may be previously subjected to a reduction treatment before hydrogenation.

The liquid phase is preferably an organic solvent. One organic solvent may be used or a mixture of two or more organic solvents may be used, but one organic solvent is preferably used alone. The organic solvent used above is not particularly limited, but examples thereof include an aliphatic hydrocarbon having 5 to 20 carbon atoms, such as dodecane or cyclohexane, an aromatic hydrocarbon having 7 to 9 carbon atoms, such as toluene or xylene, an ether having a chain structure or a cyclic structure, such as isopropyl ether, propyl ether, t-butyl methyl ether, dimethyl ether, dimethoxyethane (DME), oxetane, tetrahydrofuran (THF), methyltetrahydrofuran (MeTHF), tetrahydropyran (THP), furan, dibenzofuran, or furan, and a polyether, such as polyethylene glycol or polypropylene glycol. Among then, isopropyl ether and/or DME is preferred, and isopropyl ether is particularly preferred.

The amount of the organic solvent used is preferably, for example, in the range that gives a concentration of the amide compound of about 0.5 to 2.0% by mass. The amount of the catalyst of the present invention used based on the amount of platinum in the catalyst is preferably, for example, about 0.0001 to 50% by mole relative to the amide compound, preferably about 0.01 to 20% by mole, and more preferably about 0.1 to 5% by mole.

The catalyst of the present invention can smoothly promote hydrogenation even under mild conditions. The reaction temperature can be appropriately adjusted according to the type of the substrate or the type of the target product. For example, the reaction temperature is 150° C. or lower, preferably 10 to 100° C., more preferably about 20 to 80° C., and particularly preferably about 30 to 70° C. The pressure during reaction is 5 MPa or less, preferably a normal pressure to 4 MPa, and more preferably 2 to 3.5 MPa. The reaction time can be appropriately adjusted according to the reaction temperature and pressure, and the reaction time is, for example, about 10 minutes to 144 hours, preferably about 20 minutes to 48 hours, and particularly preferably 40 minutes to 30 hours.

By the above method, an amide compound can be hydrogenated to obtain an amine compound, and even an amine compound that is difficult to produce by a general cross coupling reaction and the like can be produced by the method of the present invention. Specifically, in the Buchwald-Hartwig reaction which is a typical example of C—N coupling, a halogenated aryl and a primary or secondary amine can be reacted in the presence of a Pd catalyst to allow the aryl group to directly bond to the N atom of the amine, but it is not possible to incorporate one or more carbon atoms or methylene chains between the N atom and the aromatic ring. However, in the above method, when the amide compound obtained by acylating the N atom of an amine is hydrogenated, as a result, a C—N bond with one or more carbon atoms or methylene chains incorporated adjacent to the N atom of the original amine can be produced. Examples of such reactions include the following: morpholine→4-cyclohexylcarbonylmorpholine→4-cyclohexylmethylmorpholine, piperidine→1-phenylacetylpiperidine→1-phenetylpiperidine, and benzylmethylamine→benzylmethylphenylacetylamide→benzylmethylphenetylamine.

In a preferred aspect of hydrogenation of an amide compound using the catalyst of the present invention, isopropyl ether is used as an organic solvent, molecular sieve or the like is placed in a vessel, and the pressure during the reaction is 4 MPa or less, the reaction time is about 10 minutes to 48 hours, and the reaction temperature is 10 to 150° C.

### (Reuse of Catalyst)

In the catalyst of the present invention, since platinum which is an active component is fixed on hydroxyapatite, the platinum fixed hardly forms larger particles even in the reaction. In addition, the catalyst of the present invention can be easily collected by, for example, a physical separation technique, such as filtration or centrifugation, from the reaction liquid after hydrogenation. The collected catalyst of the present invention can be reused as it is or after subjected to washing, drying, calcination, or the like, as required. Washing, drying, calcination, or the like may be achieved in the same manner as in the production of the catalyst of the present invention.

The collected catalyst of the present invention can exhibit the same catalytic ability as compared with the fresh catalyst of the present invention, and even after use-regeneration is repeated plural times, decrease in the catalytic ability can be significantly suppressed. Thus, according to the present invention, a catalyst which generally occupies a large proportion of the cost of hydrogenation can be collected and is repeatedly used, thus making it possible to greatly reduce the cost of hydrogenation of an amide compound.

### EXAMPLES

Hereinafter, the catalyst of the present invention and Examples of the present invention will be specifically described, but the present invention is not to be limited to the following examples, and can be widely applied within the gist of the present invention. Note that in the following examples, the surface coverage is obtained by assigning, into (Formula 1) or (Formula 3) described above, particle sizes determined by an EDS line analysis, or active surface areas or particle sizes of Pt nanoparticles in Pt/HAP and Pt—V/HAP in which the same amount of Pt is fixed which are measured using the flow-through type chemisorption measurement device described above.

### Production Example 1

### Preparation of Pt—V/HAP:

To 90 mL of acetone were added 0.4 mmol of Pt(acac)₂ manufactured by N. E. CHEMCAT CORPORATION and 0.4 mmol of VO(acac)₂ manufactured by Sigma Aldrich, and the mixture was stirred at room temperature for 30 minutes. Furthermore, 1.0 g of HAP (tradename “Tricalcium phosphate”) manufactured by Wako Pure Chemical Corporation was added thereto and the mixture was stirred at room temperature for 4 hours. The solvent was removed from the resulting mixture with a rotary evaporator to thereby obtain pale green powder. The resulting powder was dried at 110° C. overnight. Furthermore, the dried powder was pulverized in an agate mortar and was calcined in the air at 300° C. for 2 hours to thereby obtain dark grey powder (Pt—V/HAP).

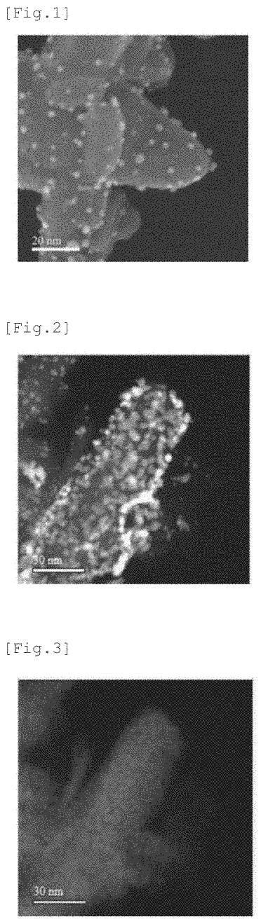

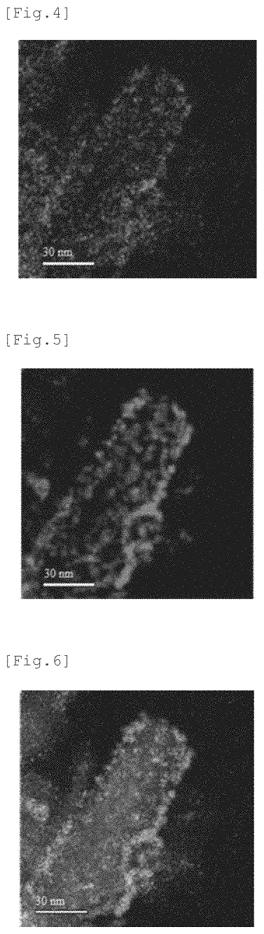

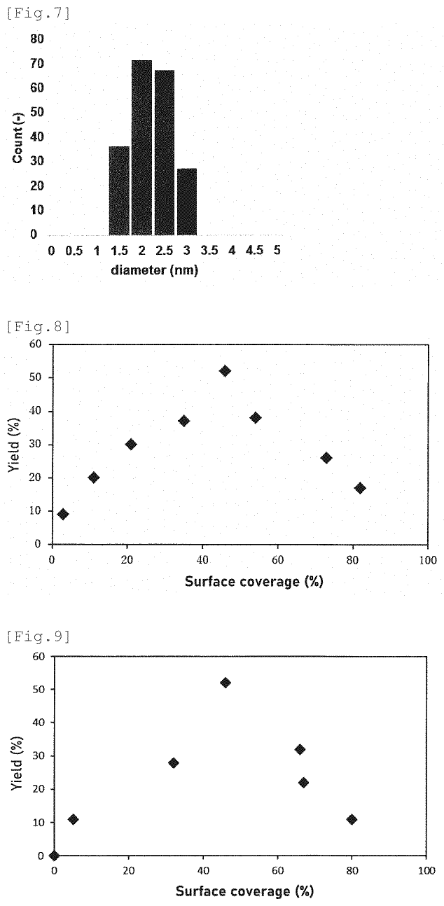

The Pt—V/HAP obtained above was subjected to various analyses. FIG. 1 and FIG. 2 respectively show a TEM image and an ADF-STEM image of the Pt—V/HAP. FIG. 3 shows a Ca element mapping image, FIG. 4 shows a V element mapping image, and FIG. 5 shows a Pt element mapping image. FIG. 6 shows a view of the Ca, V, and Pt element mapping images overlapped. It was found from the results that in the catalyst of the present invention, platinum particles were fixed on hydroxyapatite, and vanadium oxide (V₂O₅) was present in the vicinity of or on the platinum particles, with the molar ratio [Pt:V] in terms of the number of moles of platinum (Pt) as metal:number of moles of vanadium (V) as metal being 6:7 and the amount of platinum as metal being 5.8% by weight. In addition, according to the result of the EDS line analysis of Pt—V/HAP (FIG. 7), the particle size of the platinum particles was 2.2 nm. The surface coverage was 46%.

### Example 1

The catalyst obtained in Production Example 1 in an amount shown in Table 1, 5 mL of 1,2-dimethoxyethane (DME) as an organic solvent, 0.1 g of molecular sieve 4 Å manufactured by Wako Pure Chemical Corporation, and 0.5 mmol of N-acetylmorpholine as a substrate were added into a 50 mL stainless steel autoclave, and a hydrogenation reaction was performed under conditions shown in Table 1. After the reaction, the yield of 2 was measured using gas chromatography. The results are shown in Table 1.

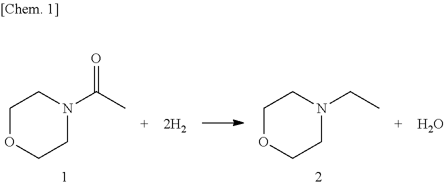

TABLE 1

| | Amount of | Reaction | Hydrogen | Reaction | |
|---|---|---|---|---|---|
| | catalyst | temperature | pressure | time | |
| Catalyst | (g) | (° C.) | (MPa) | (h) | Yield (%) |
| Pt-V/HAP | 0.1 | 70 | 3 | 1 | 70 |
| | 0.1 | 70 | 3 | 8 | 99 |
| | 0.3 | 25 | 3 | 24 | 93 |
| | 0.3 | 25 | 0.5 | 48 | 85 |
| | 0.3 | 70 | 0.1 | 48 | 95 |
| Pt/HAP | 0.1 | 70 | 3 | 1 | 0 |
| V/HAP | 0.1 | 70 | 3 | 1 | 0 |

### Example 2

The Pt—V/HAP obtained in Production Example 1 in each amount shown in Table 2, 0.5 mmol of each substrate shown in Table 2, and 0.1 g of molecular sieve 4 Å manufactured by Wako Pure Chemical Corporation were added into a 50 mL stainless steel autoclave, 5 mL of 1,2-dimethoxyethane (DME) as an organic solvent was added thereto, and a hydrogenation reaction was performed at a reaction temperature of 70° C. and a hydrogen pressure of 3 MPa. After the reaction, the yield of 4 was measured using gas chromatography. The results are shown in Table 2.

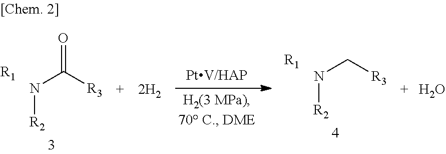

TABLE 2

| | | Amount of | Reaction | Conversion | Yield of 4 |
|---|---|---|---|---|---|
| Substrate | Product | catalyst (g) | time (h) | (%) | (%) |
| 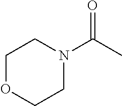 | 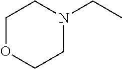 | 0.1 | 8 | >99 | 99 |
| 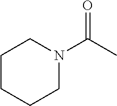 | 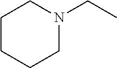 | 0.3 | 18 | 99 | 97 |
| 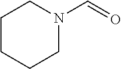 | 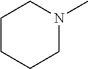 | 0.3 | 36 | 92 | 88 |
| 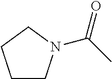 | 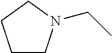 | 0.3 | 18 | 96 | 91 |
| 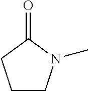 | 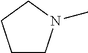 | 0.3 | 48 | 92 | 88 |
| 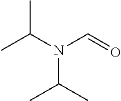 | 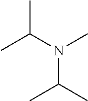 | 0.3 | 18 | 99 | 99 |
| 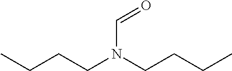 | 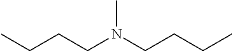 | 0.3 | 36 | 86 | 82 |
| 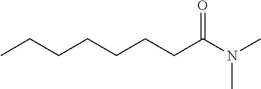 | 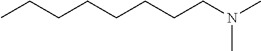 | 0.3 | 18 | 94 | 90 |
| 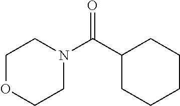 [SMILES: O=C(C1CCCCC1)N1CCOCC1] | 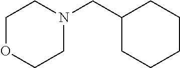 [SMILES: C1CCC(CN2CCOCC2)CC1] | 0.1 | 48 | 97 | 96 |
| 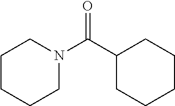 [SMILES: O=C(C1CCCCC1)N1CCCCC1] | 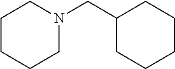 [SMILES: C1CCC(CN2CCCCC2)CC1] | 0.1 | 48 | 85 | 86 |
| 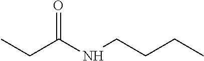 | 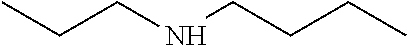 | 0.3 | 24 | 90 | 88 |
| 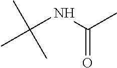 | 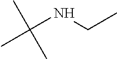 | 0.3 | 24 | 81 | 80 |
| 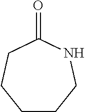 | 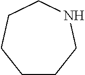 | 0.3 | 24 | 92 | 90 |
| 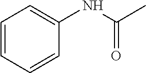 | 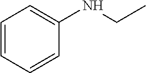 | 0.1 | 6 | 65 | 22 |
| 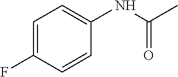 | 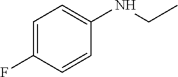 | 0.1 | 4 | 78 | 50 |
| 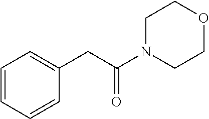 [SMILES: O=C(Cc1ccccc1)N1CCOCC1] | 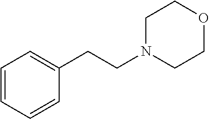 [SMILES: c1ccc(CCN2CCOCC2)cc1] | 0.1 | 8 | 99 | 96 |
| 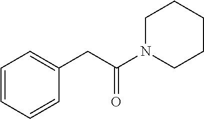 [SMILES: O=C(Cc1ccccc1)N1CCCCC1] | 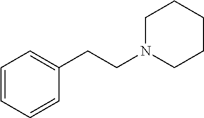 [SMILES: c1ccc(CCN2CCCCC2)cc1] | 0.1 | 24 | 90 | 88 |
| 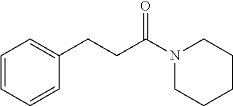 [SMILES: O=C(CCc1ccccc1)N1CCCCC1] | 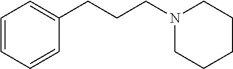 [SMILES: c1ccc(CCCN2CCCCC2)cc1] | 0.2 | 24 | 97 | 93 |
| 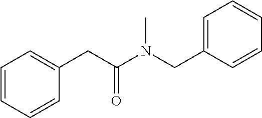 [SMILES: CN(Cc1ccccc1)C(=O)Cc1ccccc1] | 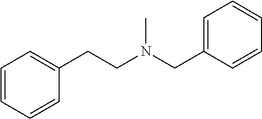 [SMILES: CN(CCc1ccccc1)Cc1ccccc1] | 0.3 | 48 | 83 | 76 |
| 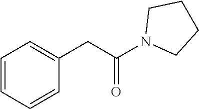 [SMILES: O=C(Cc1ccccc1)N1CCCC1] | 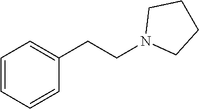 [SMILES: c1ccc(CCN2CCCC2)cc1] | 0.3 | 48 | 92 | 88 |
| 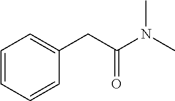 | 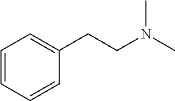 | 0.1 | 24 | 98 | 93 |

It was found that the Pt—V/HAP was able to promote a hydrogenation reaction of an amide compound under mild conditions to give a high yield also with different substrates.

### Example 3

### Reuse of Catalyst:

After the reaction of Example 1, the Pt—V/HAP used was separated by centrifugation and was washed with 1,2-dimethoxyethane (DME) as an organic solvent, whereby the Pt—V/HAP was collected from the reaction system. The collected Pt—V/HAP was used again in the same reaction. The results are shown in Table 3.

TABLE 3

| | Number of uses | Reaction time | Conversion | Yield |
|---|---|---|---|---|
| Catalyst | of catalyst | (h) | (%) | (%) |
| Pt-V/HAP | 1 | 8 | >99 | 99 |
| | 2 | 8 | 99 | 99 |
| | 3 | 8 | 99 | 99 |
| | 4 | 8 | 98 | 98 |
| | 5 | 8 | 99 | 99 |
| | 6 | 8 | 99 | 99 |
| | 7 | 8 | 98 | 98 |
| | 8 | 8 | 99 | 99 |
| | 9 | 8 | 97 | 97 |
| | 10 | 8 | 99 | 99 |

It was found that the Pt—V/HAP was able to be reused without deterioration in performance.

### Example 4

The Pt—V/HAP obtained in Production Example 1 in an amount of 0.1 g, 0.5 mmol of a substrate, and 0.1 g of molecular sieve 4 Å manufactured by Wako Pure Chemical Corporation were added into a 50 mL stainless steel autoclave, 5 mL of 1,2-dimethoxyethane (DME) as an organic solvent was added thereto, and a hydrogenation reaction was performed at each reaction temperature and hydrogen pressure shown in Table 4. After the reaction, the yield of 4 was measured using gas chromatography. The results are shown in Table 4.

TABLE 4

| | | Hydrogen | Conversion | Reaction | Yield of 4 |
|---|---|---|---|---|---|
| Substrate | Product | pressure (bar) | (%) | temperature(° C.) | (%) |
| 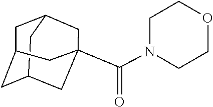 [SMILES: O=C(N1CCOCC1)C12CC3CC(CC(C3)C1)C2] | 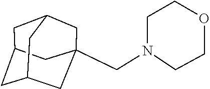 [SMILES: C1CN(CC23CC4CC(CC(C4)C2)C3)CCO1] | 1 | 100 | 99 | 97 |
| 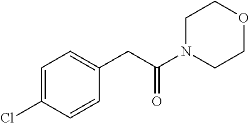 [SMILES: O=C(Cc1ccc(Cl)cc1)N1CCOCC1] | 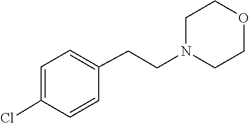 [SMILES: Clc1ccc(CCN2CCOCC2)cc1] | 30 | Room temperature | 96 | >99 |

It was found that the Pt—V/HAP was able to promote a hydrogenation reaction of an amide compound under mild conditions to give a high yield also with different substrates, different hydrogen pressures, and different reaction temperatures.

### Production Example 2

Preparation of Pt—V/HAP with Constant Amount of Pt and Different Amounts of V:

Pt—V/HAP having various amounts of vanadium was produced in the same manner except that the amount of VO(acac)₂ in Production Example 1 was changed to 0.025, 0.05, 0.1, 0.2, 0.6, 0.8, or 1.0 mmol. The surface coverages of the catalysts are determined using a flow-through type chemisorption measurement device. The results are shown in Table 5.

TABLE 5

| | Production Example 2 | | | | | | |
|---|---|---|---|---|---|---|---|
| Amount of V | 0.025 | 0.05 | 0.1 | 0.2 | 0.6 | 0.8 | 1.0 |
| charged | | | | | | | |
| (mmol/g) | | | | | | | |
| Amount of Pt | 0.4 | 0.4 | 0.4 | 0.4 | 0.4 | 0.4 | 0.4 |
| charged | | | | | | | |
| (mmol/g) | | | | | | | |
| Surface | 3 | 11 | 21 | 35 | 46 | 54 | 73 |
| coverage (%) | | | | | | | |

### Production Example 3

### Preparation of Pt—V/HAP Having Constant Pt:V Ratio and Different Total Amounts of Pt and V:

Pt—V/HAP having various amounts of each metal was obtained in the same manner except that, in Production Example 1, the ratio of the Pt(acac)₂ and VO(acac)₂ was fixed to 1:1 and the amounts of platinum and vanadium were the same amount of 0.1, 0.2, 0.6, 0.8, or 1.0 mmol. The surface coverages of the catalysts were determined using a flow-through type chemisorption measurement device. The results are shown in Table 6.

TABLE 6

| | Production Example 3 | | | | |
|---|---|---|---|---|---|
| Amount of V charged (mmol/g) | 0.1 | 0.2 | 0.6 | 0.8 | 1.0 |
| Amount of Pt charged (mmol/g) | 0.1 | 0.2 | 0.6 | 0.8 | 1.0 |
| Surface coverage (%) | 5 | 32 | 68 | 67 | 80 |

### Production Example 4

### Preparation of Mixture of Pt/HAP and V/HAP:

### (1) Preparation of Pt/HAP

Pt/HAP was obtained in the same manner as in Production Example 1 except that VO(acac)₂ was not used.

### (2) Preparation of V/HAP

V/HAP was obtained in the same manner as in Production Example 1 except that Pt(acac)₂ was not used.

### (3) Preparation of Mixture of Pt/HAP and V/HAP

Pt/HAP prepared in (1) and V/HAP prepared in (2) were mixed in the same amount of 0.05 or 0.2 mmol to obtain a mixture of Pt/HAP and V/HAP. The surface coverages of the catalysts were determined using a flow-through type chemisorption measurement device. The results are shown in Table 7.

TABLE 7

| | Production Example 4 | |
|---|---|---|
| Amount of V charged (mmol/g) | 0.05 | 0.2 |
| Amount of Pt charged (mmol/g) | 0.05 | 0.2 |
| Surface coverage (%) | 0 | 0 |

### Example 5

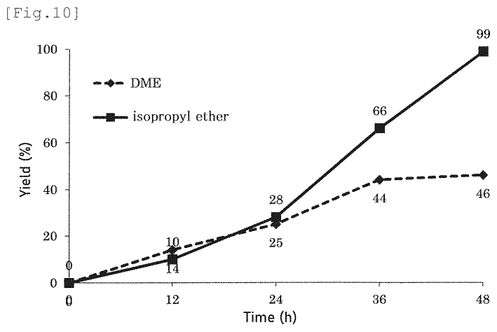

For each of the catalysts obtained in Production Examples 1 and 2, a reaction is performed in the same manner as in Example 1 except that a hydrogenation reaction is performed using 0.1 g of molecular sieve 4 Å manufactured by Wako Pure Chemical Corporation with an amount of platinum of 3 mol % relative to the substrate at a hydrogen pressure of 3 MPa and a temperature of 70° C. for 30 minutes. After the reaction, the yield of 2 was measured using gas chromatography. The results are shown in Table 8. The relationship between the surface coverage and the yield was shown in FIG. 8.

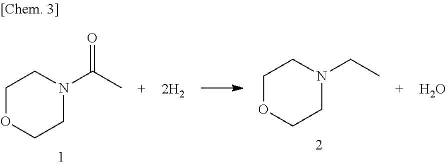

TABLE 8

| | Production | | | | Production | Production | | |
|---|---|---|---|---|---|---|---|---|
| | Example 2 | | | | Example 1 | Example 2 | | |
| Surface | 3 | 11 | 21 | 35 | 46 | 54 | 73 | 82 |
| coverage (%) | | | | | | | | |
| Yield (%) | 9 | 20 | 30 | 37 | 52 | 38 | 26 | 17 |

### Example 6

For each of the catalysts obtained in Production Examples 1, 3, and 4, a hydrogenation reaction was performed in the same manner as in Example 5. After the reaction, the yield of 2 was measured using gas chromatography. The results are shown in Table 9. The relationship between the surface coverage and the yield was shown in FIG. 9.

TABLE 9

| | Production | Production | | Production | Production | Production | | |
|---|---|---|---|---|---|---|---|---|
| | Example 4*¹ | Example 3 | | Example 4*² | Example 1 | Example 3 | | |
| Surface | 0 | 5 | 32 | 0 | 46 | 68 | 67 | 80 |
| coverage (%) | | | | | | | | |
| Yield (%) | 0 | 11 | 28 | 0 | 52 | 32 | 22 | 11 |

*¹Mixture of amount of V charged of 0.05 mmol/g and amount of Pt charged of 0.05 mmol/g
*²Mixture of amount of V charged of 0.2 mmol/g and amount of Pt charged of 0.2 mmol/g

It was found from Table 8 and FIG. 8 that although the amounts of metals are different, there is an optimal range in the coverage of platinum with vanadium. On the other hand, it was found from Table 9 and FIG. 9 that although the total amount of metals (fixing concentration) was different, there is an optimal range in the coverage of platinum with vanadium also in this case.

In addition, a catalyst having a higher concentration of a catalytically active species, such as platinum, tends to have poorer dispersibility of the active species on hydroxyapatite as compared with a catalyst having a lower concentration of the active species. The catalyst having poorer dispersibility of the active species tends to have poorer catalytic activity even with the same amount of the active species. In Table 9, the [PT:V=1.0:1.0] catalyst of Production Example 3, in contrast to the [PT:V=0.4:0.4] catalyst of Production Example 1, has a concern to give a reduced yield with the concentration of the activity species being a negative element.

Thus, for the [PT:V=1.0:1.0] catalyst of Production Example 3 and the [PT:V=0.4:0.4] catalyst of Production Example 1, the yields were measured under the same conditions as in Example 5 with the same total weight of the catalyst including the hydroxyapatite. The results are shown in Table 10.

TABLE 10

| | Production | Production |
|---|---|---|
| | Example 1 | Example 3 |
| Amount of V charged (mmol/g) | 0.4 | 1.0 |
| Amount of Pt charged (mmol/g) | 0.4 | 1.0 |
| Amount of catalyst (g) | 0.1 | 0.1 |
| Surface coverage (%) | 46 | 80 |
| Yield (%) | 52 | 28 |

In a hydrogenation reaction using the catalyst of Production Example 3, the amounts of platinum and vanadium which were active species in the catalyst were increased to twice or more, and the yield was increased as compared with the evaluation in Table 9 in which the same catalyst was used. However, it was found that the yield was decreased as compared with the catalyst of Production Example 1 in spite of the larger amount of platinum.

### Example 7

A hydrogen reaction was performed in the same manner as in Example 1 using 0.1 g of the catalyst obtained in Production Example 1, 0.5 mmol of N-acetylmorpholine as a substrate, and 0.1 g of molecular sieve 4 Å manufactured by Wako Pure Chemical Corporation and using a solvent shown in Table 11 as an organic solvent with an amount of platinum relative to the substrate of 6 mol % at a hydrogen pressure of 3 MPa and a temperature of 70° C. for 1 hour. After the reaction, the yield of 2 was measured using gas chromatography. The results are shown in Table 11.

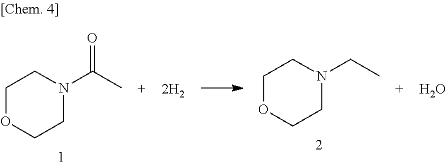

TABLE 11

| Organic solvent | Yield [%] |
|---|---|
| 1,2-Dimethoxyethane (DME) | 70 |
| 2-Methyltetrahydrofuran (MeTHF) | 57 |
| Cyclopentyl methyl ether (CPME) | 67 |
| t-Amyl methyl ether (TAME) | 55 |
| Isopropyl ether | 98 |
| t-Butyl methyl ether | 78 |
| Propyl ether | 66 |
| Diisopropyl amine | 30 |
| 2,4-Dimethylpentane | 20 |

It was found from the results that in hydrogenation of amide compounds in which the catalyst of the present invention was used, by using isopropyl ether as an organic solvent, the yields of the amine compounds were significantly increased.

### Example 8

Into a 50 mL stainless steel autoclave were added 0.15 g of the catalyst obtained in Production Example 1, 5 mL of isopropyl ether as an organic solvent, 0.25 mmol of an amide compound shown in Table 12 as a substrate, and 0.2 g of molecular sieve 4 Å manufactured by Wako Pure Chemical Corporation, and a hydrogenation reaction was performed with an amount of platinum relative to the substrate of 18 mol % at a hydrogen pressure of 0.1 MPa and a temperature shown in Table 12 for 48 hours. After the reaction, the yield of the amine compound was measured using gas chromatography. The results are shown in Table 12.

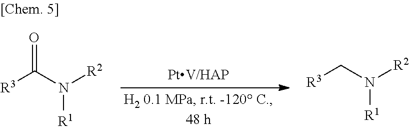

TABLE 12

| Substrate | Product | Temperature [° C.] | Yield [%] |
|---|---|---|---|
| 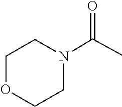 | 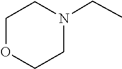 | r.t. | 99 |
| 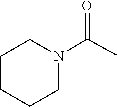 | 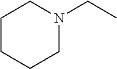 | 100 | 99 |
| 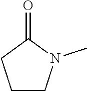 | 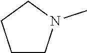 | 70 r.t. | 99 75 |
| 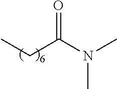 | 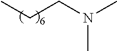 | 120 | 99 |
| 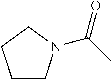 | 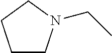 | 70 r.t. | 99 99 |
| 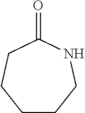 | 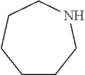 | 120 | 79 |
| 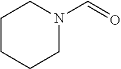 | 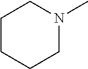 | 120 | 99 |
| 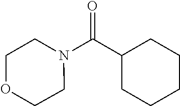 [SMILES: O=C(C1CCCCC1)N1CCOCC1] | 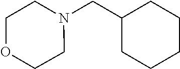 [SMILES: C1CCC(CN2CCOCC2)CC1] | 70 | 65 |
| 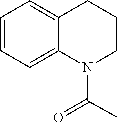 | 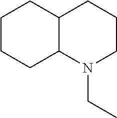 | 120 | 78 |
| 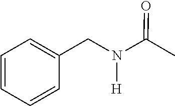 | 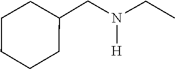 | 70 | 45 |
| 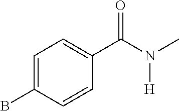 | 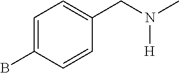 | 100 | 63 |
| 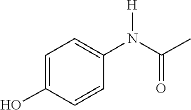 | 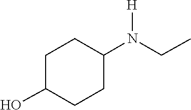 | 100 | 45 |
| 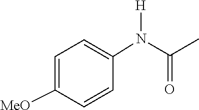 | 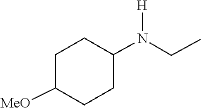 | 100 | 56 |

It was found from the results that in hydrogenation reactions using the catalyst of the present invention, by using isopropyl ether as an organic solvent, amine compounds were able to be obtained at high yields even under a pressure of hydrogen gas of 0.1 MPa which is as low as the atmospheric pressure.

### Example 9

Hydrogenation of imides was performed in the same manner as in Example 8 except that the pressure of hydrogen gas, the reaction temperature, and the reaction time were changed as shown in Table 13. After the reaction, the yield of the amine compound was measured using gas chromatography. The results are shown in Table 13.

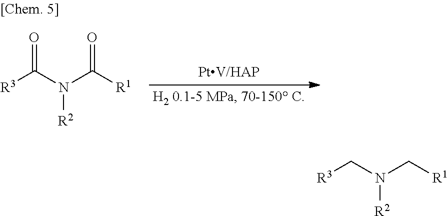

TABLE 13

| | | H₂/temperature/time | Yield | Conversion |
|---|---|---|---|---|
| Substrate | Product | [MPa]/[° C.]/[h] | [%] | [%] |
| 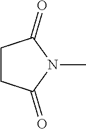 | 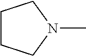 | 3/70/48 0.1/70/48 | 99 99 | 99 99 |
| 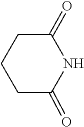 | 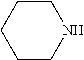 | 3/150/48 | 79 | 90 |
| 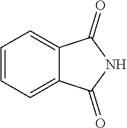 [SMILES: O=C1NC(=O)c2ccccc21] | 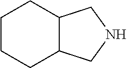 | 3/110/48 | 77 | 99 |
| 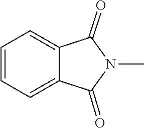 | 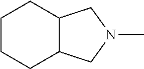 | 3/110/48 | 83 | 99 |
| 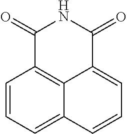 [SMILES: O=C1NC(=O)c2cccc3cccc1c23] | 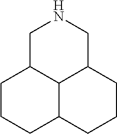 [SMILES: C1CC2CCCC3CNCC(C1)C23] | 5/150/72 | 44 | 77 |
| 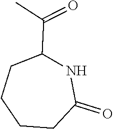 | 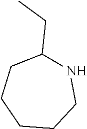 | 5/150/48 | 55 | 99 |
| 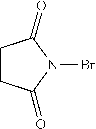 | 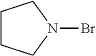 | 3/70/48 | 88 | 99 |

It was found from the results that by using isopropyl ether as an organic solvent, the catalyst of the present invention was able to achieve excellent conversion ratios and yields also in hydrogenation of imide compounds. While a pressure of hydrogen gas exceeding 10 MPa is generally required for hydrogenation of imide compounds, it was found that the catalyst of the present invention can promote hydrogenation of imide compounds under such a low pressure as above.

### Example 10

A hydrogenation reaction at room temperature was performed in the same manner as in Example 1 except that 0.15 g of the catalyst obtained in Production Example 1, 0.25 mmol of N-acetylmorpholine as a substrate, 0.1 MPa of a hydrogen pressure, a room temperature of a reaction temperature, and 0.2 g of molecular sieve 4 Å manufactured by Wako Pure Chemical Corporation were used, and that the organic solvent was changed to DME or isopropyl ether. The yields of the amine compound at a reaction time of 0 to 48 hours were measured using gas chromatography. The results are shown in FIG. 10.

It was found from the results that by using isopropyl ether as an organic solvent, an amine compound was able to be obtained with a high yield.

### INDUSTRIAL APPLICABILITY

The catalyst of the present invention is useful for safely producing amino compounds which are useful in medicine, agrochemical, or other various industrial fields under mild conditions. In addition, the catalyst of the present invention can be produced in an inexpensive and safe manner.

1. A hydrogenation catalyst for an amide compound, the catalyst comprising
hydroxyapatite, and
platinum and vanadium that are fixed on the hydroxyapatite,
15 to 80% of the surface of the platinum being covered with vanadium.

2. The hydrogenation catalyst for an amide compound according to claim 1, wherein the amide compound is a secondary or higher amide compound or an amide compound having an aromatic substituent.

3. A method for producing an amine compound, the method comprising bringing an amide compound into contact with the hydrogenation catalyst for an amide compound according to claim 1 to perform hydrogenation of the amide compound, thus producing an amine compound.

4. The method for producing an amine compound according to claim 3, wherein the hydrogenation is performed at 100° C. or lower.

5. The method for producing an amine compound according to claim 3, wherein the hydrogenation is performed at 5 MPa or less.

6. The method for producing an amine compound according to claim 3, wherein the amide compound is a secondary or higher amide compound or an amide compound having an aromatic substituent.

7. The method for producing an amine compound according claim 3, wherein the hydrogenation is performed in a liquid phase.

8. The method for producing an amine compound according to claim 7, wherein the liquid phase is an organic solvent.

9. The method for producing an amine compound according to claim 8, wherein the organic solvent is isopropyl ether.

10. A method for producing hydrogenation catalyst for an amide compound according to claim 1, the method comprising fixing platinum and vanadium on hydroxyapatite in a solvent, and then drying the resultant.

11. The method for producing hydrogenation catalyst for an amide compound according to claim 10, wherein hydroxyapatite and a solvent mixture liquid containing a platinum compound and a vanadium compound are mixed to fix platinum and vanadium on the hydroxyapatite in the solvent.

12. The method for producing hydrogenation catalyst for an amide compound according to claim 10, wherein hydroxyapatite, a solvent liquid containing a platinum compound, and a solvent liquid containing a vanadium compound are mixed in any order to fix platinum and vanadium on the hydroxyapatite in the solvent.

13. The method for producing hydrogenation catalyst for an amide compound according to claim 10, wherein the solvent is water.
